(19) United States
(12) Patent Application Publication
(10) Pub. No.: US 2024/0074940 A1
(43) Pub. Date: Mar. 7, 2024

(54) Ultrasonic massage device applying synchronic treatment to multiple parts of human body

(71) Applicant: Zhijun PENG, Foshan (CN)

(72) Inventor: Zhijun PENG, Foshan (CN)

(21) Appl. No.: 18/123,343

(22) Filed: Mar. 20, 2023

(30) Foreign Application Priority Data
Sep. 7, 2022 (CN) 202211088296.8

(51) Int. Cl. A61H 23/02

(52) U.S. Cl. CPC A61H 23/0245

(57) ABSTRACT

An ultrasonic massage device applying synchronic treatment to multiple parts of human body, including a host, an ultrasonic vibrating motor built in the host, a key panel controlling the ultrasonic vibrating motor, and a plurality of massage heads installed on different sides of the host; the ultrasonic vibrating motor produces and transmits high-frequency ultrasonic vibration to the plurality of massage heads to provide synchronic massage to various body parts. The host with built-in ultrasonic vibrating motor is connected with massage heads at different positions and with different orientations; when the ultrasonic vibrating motor is operating, high-frequency ultrasonic vibration is generated and the massage heads vibrate synchronically. Different parts of human body then receive synchronic massage treatment when in contact with the massage heads.

### FIELD OF THE INVENTION

The present invention relates to the field of therapeutic massage devices, and more particularly an ultrasonic therapeutic massage device.

### BACKGROUND OF THE INVENTION

The present small-sized therapeutic massage devices adopting high-frequency ultrasonic motors are only designed for a certain part of the human body. Such devices only provide massage to a certain part of the human body but not simultaneous massage to multiple parts of the human body. Examples include a massage device applying neurological therapeutic treatment to head and neck (Chinese Utility Patent No.: CN216908580U), an easy-to-use leg massage device (Chinese Utility Patent No.: CN216877118U), and a wrist support with an openable curved metal plate (Chinese Utility Patent No.: CN216753607U). These disclosed technical solutions are only developed and designed to fit a certain part of the human body. To massage multiple body parts, it costs more to choose and purchase different massage devices. It is also time-consuming for body parts to receive massage one by one. The design drawbacks and the limited scope of application of the present ultrasonic massage devices cause wastage of money and time, and hence such devices shall be redesigned and modified to better suit the need of users.

### BRIEF SUMMARY OF THE INVENTION

To solve the aforementioned problems, the present invention provides an ultrasonic massage device applying synchronic treatment to multiple parts of human body, wherein a host with a built-in ultrasonic vibrating motor is connected with massage heads at different positions and with different orientations; when the ultrasonic vibrating motor is operating, high-frequency ultrasonic vibration is generated and the massage heads vibrate synchronically. Different parts of human body then receive synchronic massage treatment when in contact with the massage heads. This allows user to receive massage treatment for more body parts in limited time, and hence saves time and increases the efficiency of massage. The present invention also promotes blood circulation and metabolism, and maintains the general health of the human body, and is easy to use, simple in structure, reliable and durable.

The technical solution is as follows: the present invention provides an ultrasonic massage device applying synchronic treatment to multiple parts of human body, comprising a host, an ultrasonic vibrating motor built in the host, a key panel controlling the ultrasonic vibrating motor, and a plurality of massage heads installed on different sides of the host; the ultrasonic vibrating motor produces and transmits high-frequency ultrasonic vibration to the plurality of massage heads to provide synchronic massage to various body parts.

Further, one of the massage heads is installed on a top surface of the host, another two massage heads are installed on a bottom surface of the host, and still another one of the massage heads is installed on a back surface of the host.

Further, a telescopic connecting rod is provided between each massage head and the host.

Further, the host comprises a main case, a cover, a storage battery and a mainboard; a mounting seat is provided at a center of the main case; the ultrasonic vibrating motor is fixed in the mounting seat; the storage battery and the mainboard are installed in the main case but outside the mounting seat; the cover is installed on the main case; the key panel is provided on the cover and is connected with the mainboard.

The benefits of the present invention are as follows: A host with a built-in ultrasonic vibrating motor is connected with massage heads at different positions and with different orientations; when the ultrasonic vibrating motor is operating, high-frequency ultrasonic vibration is generated and the massage heads vibrate synchronically. Different parts of human body then receive synchronic massage treatment when in contact with the massage heads. This allows user to receive massage treatment for more body parts in limited time, and hence saves time and increases the efficiency of massage. The present invention also promotes blood circulation and metabolism and maintains the general health of the human body. Unlike the inefficient and time-consuming prior art which is only applicable to one certain body part, the present invention solves these problems. The massage heads may be designed to fit different body parts and provide synchronic massage to multiple body parts. The present invention is easy to use, simple in structure, reliable and durable. Moreover, only one high-frequency vibration motor is required, which is power-saving and light in weight.

### BRIEF DESCRIPTION OF THE DRAWINGS

FIG. 1 shows a first structural view of the present invention;

FIG. 2 shows a second structural view of the present invention;

FIG. 3 shows an partially exploded perspective view of the present invention;

FIG. 4 shows a first structural view of the telescopic connecting rod and a massage head of the present invention;

FIG. 5 shows a second structural view of the telescopic connecting rod and another massage head of the present invention.

### DETAILED DESCRIPTION OF THE INVENTION

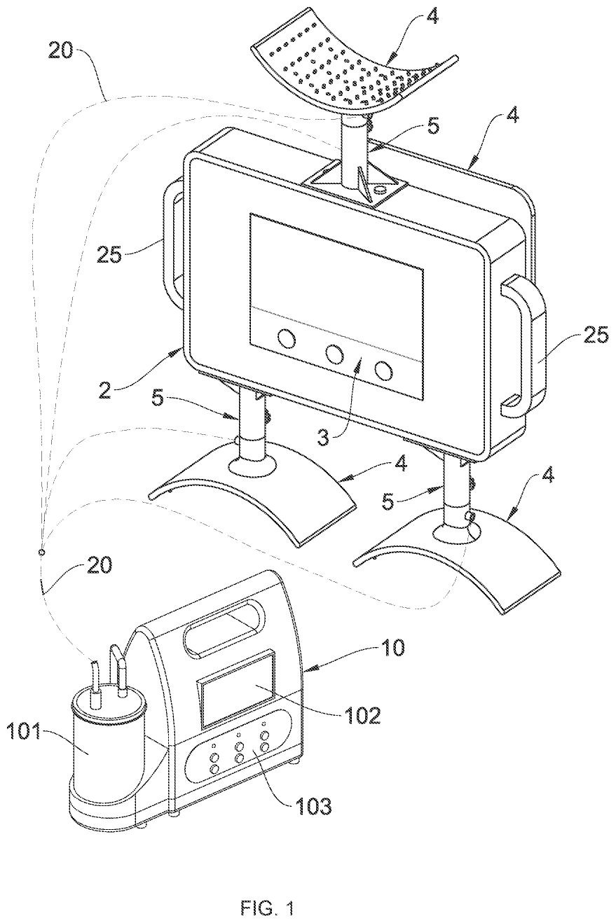

As illustrated in FIG. 1 to FIG. 3, the present invention provides an ultrasonic massage device applying synchronic treatment to multiple parts of human body, comprising: a host 2, an ultrasonic vibrating motor 1 built in the host 2, a key panel 3 controlling the ultrasonic vibrating motor 1, and a plurality of massage heads 4 respectively installed on different sides of the host 2; the ultrasonic vibrating motor 1 produces and transmits high-frequency ultrasonic vibration to the plurality of massage heads 4 to provide synchronic massage to various body parts. In this embodiment, one of the massage heads 4 is installed on a top surface of the host 2, another two of the massage heads 4 are installed on a bottom surface of the host 2, and still another one of the massage heads 4 is installed on a back surface of the host 2. When in use, the user sits on a sofa or a chair; the two massage heads 4 installed on the bottom surface press on the thighs respectively, while the massage head 4 installed on the back surface presses against the abdomen and the massage head 4 installed on the top surface presses against the jaw; the ultrasonic vibrating motor 1 operates and synchronically transmits high-frequency ultrasonic vibration to each massage head 4 to massage the thighs, the abdomen and the jaw at the same time.

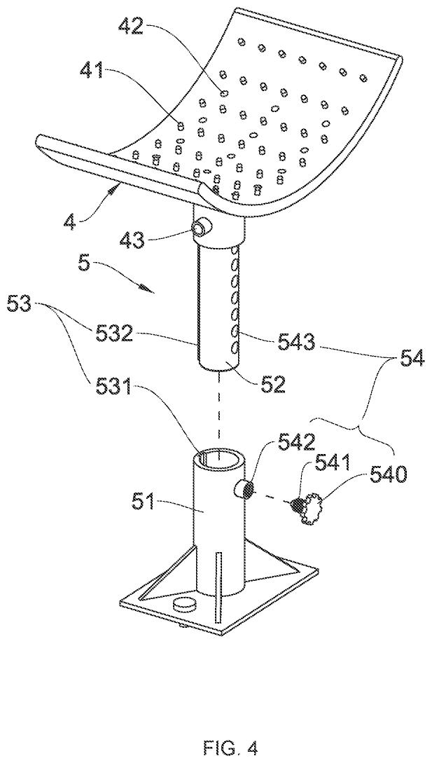

To adjust the position of each massage head 4, a telescopic connecting rod 5 is provided between each massage head 4 and the host 2. As illustrated in FIG. 4, the telescopic connecting rod 5 comprises a main rod 51 sleeving an auxiliary rod 52; the main rod 51 is a hollow rod while the auxiliary rod 52 may be a solid or a hollow rod. A limiting structure 53 and a locking structure 54 are provided between the main rod 51 and the auxiliary rod 52. The limiting structure 53 fixes a direction of movement of the auxiliary rod 52 in the main rod 51 and prevents the auxiliary rod 52 from circumferential rotation in the main rod 51; the locking structure 54 locks the auxiliary rod 52 from further moving with respect to the main rod 51 when the auxiliary rod 52 reaches an appropriate position with respect to the main rod 51 after the auxiliary rod 52 is pulled out from or retracted into the main rod 51. As illustrated from FIG. 1 to FIG. 3, each massage head 4 is connected to a respective auxiliary rod 52 and each main rod 51 is connected to the host 2.

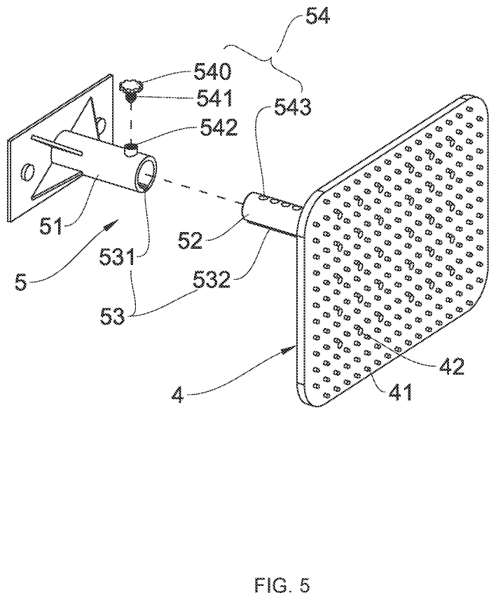

As illustrated in FIG. 4 and FIG. 5, the limiting structure 53 comprises a guiding groove 531 and a guiding protruding strip 532. In this embodiment, the guiding groove 531 is provided on an inner wall of the main rod 51, and the guiding protruding strip 532 is provided on an outer wall of the auxiliary rod 52. The positions of the guiding groove 531 and the guiding protruding strip 532 are interchangeable.

As illustrated in FIG. 4 and FIG. 5, the locking structure 54 comprises a locking bolt 541 with a handle 540, a screw hole 542 and fixed holes 543; the screw hole 542 is provided on the main rod 51 and is in communication with an inner cavity of the main rod 51; the fixed holes 543 are provided on the outer wall of the auxiliary rod 52 and are provided in linear arrangement along a lengthwise direction of the auxiliary rod 52, such that the auxiliary rod 52 may be adjusted to and fixed in position with respect to the main rod 51.

As illustrated in FIG. 4 and FIG. 5, the massage head 4 can be made in different shapes according to actual need. For example, the massage head 4 may be in arc shape, a flat circular shape or a flat rectangular shape. The shape and structure of the massage head 4 may be designed to fit different human body parts. Massage bumps 41 are evenly distributed on a massage side of the massage head 4. Medicine-spraying holes 42 are provided between the massage bumps 41 on the massage head 4, and a pipe joint 43 is provided at a back of the massage head 4. As illustrated in FIG. 1, in a specific embodiment, an electric dosing pump 10 or a medicine-atomizing machine may be provided; the electric dosing pump 10 is connected with each pipe joint 43 through a soft hose 20; medicine solution in a liquid storage tank 101 of the electric dosing pump 10 is pumped to each massage head 4 and sprayed to human skin from the medicine-spraying holes 42. This allows massage and medicine-spraying to be carried out at the same time, hence human skin better absorbs the medicine and the therapeutic effect is better. The electric dosing pump 10 comprises a display screen 102 and control keys 103 for ease of use.

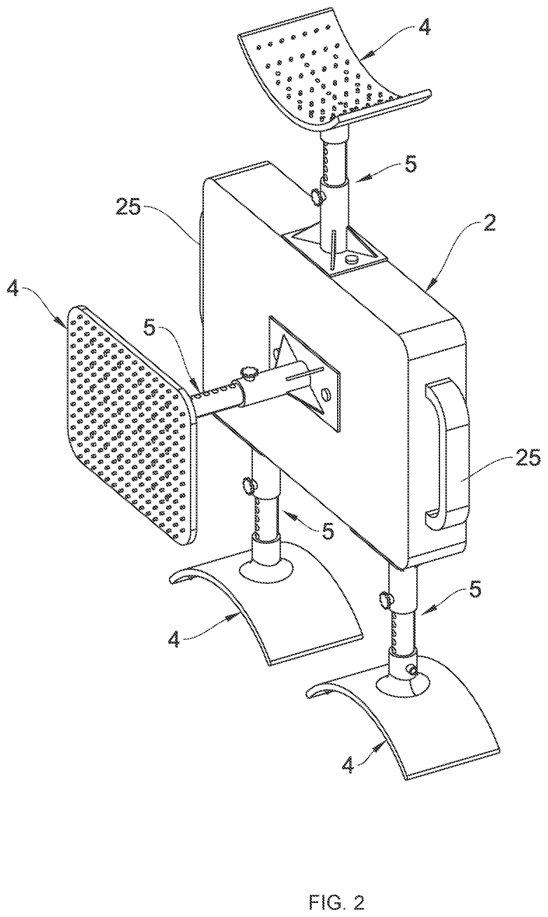

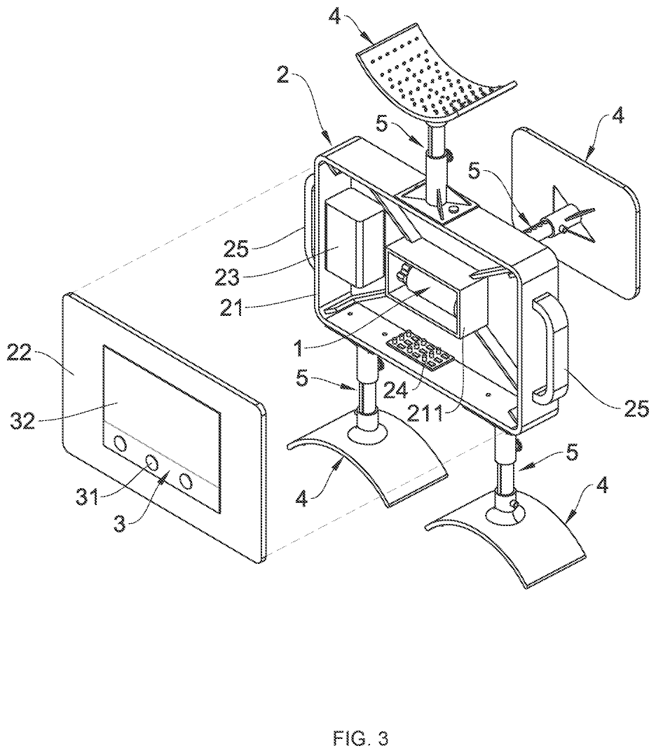

As illustrated in FIG. 3, the host 2 is simple in structure, comprising a main case 21, a cover 22, a storage battery 23 and a mainboard 24; a mounting seat 211 is provided at a center of the main case 21; the ultrasonic vibrating motor 1 is fixed in the mounting seat 211; the storage battery 23 and the mainboard 24 are installed in the main case 21 but outside the mounting seat 211; the cover 22 is installed on the main case 21; the key panel 3 is provided on the cover 22 and is connected with the mainboard 24. The key panel 3 is provided with press keys or touch keys 31 and a LCD monitor 32 for ease of use. For ease of hand-carrying the host 2, as illustrated in FIG. 1 and FIG. 2, each of two lateral sides of the main case 21 is respectively provided with a handgrip 25. The mainboard 24 may be provided with a Bluetooth® communication module or a WIFI® module operated with corresponding controlling application software installed on smartphones, so as to control the operation of the present invention with smartphones.

What is claimed is:

1. An ultrasonic massage device, comprising a host (2), an ultrasonic vibrating motor (1) built in the host (2), a key panel (3) controlling the ultrasonic vibrating motor (1), and a plurality of massage heads (4) installed on different sides of the host (2); the ultrasonic vibrating motor (1) produces and transmits high-frequency ultrasonic vibration to the plurality of massage head (4) to provide synchronic massage.

2. The ultrasonic massage device of claim 1, wherein one of the massage heads (4) is installed on a top surface of the host (2), another two of the massage heads (4) are installed on a bottom surface of the host (2), and still another one of the massage heads (4) is installed on a back surface of the host (2).

3. The ultrasonic massage device of claim 2, wherein a telescopic connecting rod (5) is provided between each massage head (4) and the host (2).

4. The ultrasonic massage device of claim 3, wherein the telescopic connecting rod (5) comprises a main rod (51) sleeving an auxiliary rod (52); a limiting structure (53) and a locking structure (54) are provided between the main rod (51) and the auxiliary rod (52); each massage head (4) is connected to a respective auxiliary rod (52) and each main rod (51) is connected to the host (2).

5. The ultrasonic massage device of claim 4, wherein the limiting structure (53) comprises a guiding groove (531) and a guiding protruding strip (532).

6. The ultrasonic massage device of claim 4, wherein the locking structure (54) comprises a locking bolt (541); a handle (540) disposed on the locking bolt (541), a screw hole (542) and fixed holes (543).

7. The ultrasonic massage device of claim 1, wherein each massage head (4) is in an arc shape or flat rectangular shape; massage bumps (41) are evenly distributed on a massage side of each massage head (4).

8. The ultrasonic massage device of claim 7, wherein medicine-spraying holes (42) are provided between the massage bumps (41) on each massage head (4), and a pipe joint (43) is provided at a back of each massage head (4).

9. The ultrasonic massage device of claim 1, wherein the host (2) comprises a main case (21), a cover (22), a storage battery (23) and a mainboard (24); a mounting seat (211) is provided at a center of the main case (21); the ultrasonic vibrating motor (1) is fixed in the mounting seat (211); the storage battery (23) and the mainboard (24) are installed in the main case (21) but outside the mounting seat (211); the cover (22) is installed on the main case (21); the key panel (3) is provided on the cover (22) and is connected with the mainboard (24).

10. The ultrasonic massage device of claim 9, wherein each of two lateral sides of the main case (21) is respectively provided with a handgrip (25).
